(19) United States
(12) Patent Application Publication
(10) Pub. No.: US 2009/0326441 A1
(43) Pub. Date: Dec. 31, 2009

(54) Ultrasonic Enhanced Microneedles

(75) Inventors: Ciprian Iliescu, Nanos (SG); Bantao Chen, Nanos (SG)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: AGENCY FOR SCIENCE ,TECHNOLOGY AND RESEARCH

(21) Appl. No.: 12/375,631

(22) PCT Filed: Aug. 1, 2007
(86) PCT No.: PCT/SG2007/000227

§ 371 Date: Sep. 2, 2009

Related U.S. Application Data
(60) Provisional application No. 60/835,059, filed on Aug. 1, 2006.

(51) Int. Cl. A61M 5/158

(52) U.S. Cl. 604/22

(57) ABSTRACT

The invention provides an injection device for injecting a substance into a subject. The device comprises an injector and an enhancer for enhancing the rate of penetration of the substance into the subject. The injector comprises a microneedle support and at least one microneedle extending from a first surface of said support. The or each microneedle has a fluid channel extending through the microneedle and through the support. The fluid channel of the or each microneedle has an inlet aperture in a second surface of the support and an outlet aperture in the microneedle.

### TECHNICAL FIELD

The present invention relates to microneedles, and arrays thereof, with enhanced drug delivery capability.

### BACKGROUND OF THE INVENTION

Numerous sophisticated and potent drugs (protein-based, DNA-based or therapeutic compounds) have been produced in the battle with disease and illness, but many of these drug compounds cannot be effectively assimilated by the body through oral medication or injections due to biological barriers in the body (e.g. the skin, the oral mucosa, the blood-brain barrier).

Transdermal delivery of drugs is an attractive option to deliver drugs or biological compounds into the human body, but relies on the diffusion of drugs across the skin and is limited by the low permeability of the skin. The rate of diffusion depends in part on the size and hydrophilicity of the drug molecules and the concentration gradient across the stratum corneum and epidermis. Although there are many potential advantages of transdermal drug delivery, it is severely limited by the poor permeability of human skin. Most drugs do not permeate the skin at therapeutically relevant levels.

Another disadvantage of transdermal drug delivery is the slow rate of drug delivery: delivery of the drug may take a long time, up to hours or days, and this may not be convenient or practical for clinical application. A number of methods have been developed to increase the rate of transdermal transport across the skin. These include use of chemical enhancers, iontophoresis, electroporation and ultrasound, however these methods have had varied levels of success in drug delivery applications.

Microneedle devices have been developed for controlled transdermal drug or biological fluid delivery in a minimum invasive, painless, and convenient manner. Transdermal drug delivery using a microneedle array enhances the rate of transport of molecules across skin by 3 to 4 orders of magnitude. In this technique micro-sized needles are used to penetrate the primary biological barrier of transdermal drug delivery, the stratum corneum, without penetrating into the dermis layer that contains nerves and blood vessels, so as to avoid the pain and bleeding. Since the stratum corneum has a depth of 10 to 20 μm, and epidermis has a variable depth of 50 to 100 μm, the penetrated length of the microneedles is commonly around 100 μm. Besides the high permeability and painless piercing, microneedle arrays have the intrinsic advantage of delivery uniformity. Another advantage of microneedle arrays is that microfabrication technology readily produces micron-level structures in a way that can be easily scaled up for cheap and reproducible mass production.

A disadvantage with use of microneedle arrays for drug delivery is that few drugs have the necessary physiochemical properties to be effectively delivered through the skin by passive diffusion. The limitations of the transdermal delivery of drugs using microneedles array are imposed by the diffusion of the drug through the epidermis. Since the delivery of a drug a diffusion phenomenon, there is a limitation of the size of macromolecule that can be delivered. Additionally, the quantity of drug that can be delivering is limited by the achieving of “saturation” value.

### OBJECT OF THE INVENTION

It is the object of the present invention to substantially overcome or at least ameliorate one or more of the above disadvantages.

### SUMMARY OF THE INVENTION

In a first aspect of the invention there is provided an injection device for injecting a substance into a subject, said injection device comprising:
(i) an injector comprising a microneedle support and at least one microneedle extending from a first surface of said support, wherein the or each microneedle has a fluid channel extending through the microneedle and through the support, the fluid channel of the or each microneedle having an inlet aperture in a second surface of the support and an outlet aperture in the microneedle; and
(ii) an enhancer for enhancing the rate of penetration of the substance into the subject.

The following options may be used as part of the first aspect, and may be used independently or in any practical combination.

The enhancer may comprise an ultrasound generator. The ultrasound generator may comprise a piezoelectric crystal. The piezoelectric crystal may comprise a PZT membrane. The piezoelectric crystal may be coupled to a source of alternating current whereby the piezoelectric crystal is capable of generating ultrasound in response to an alternating current from said source.

The injection device may additionally comprise a reservoir for containing a fluid comprising the substance. The reservoir may be disposed so as to allow the fluid to pass from the reservoir into the or each fluid channel through the inlet aperture thereof. The enhancer may be coupled to the microneedle support so as to define the reservoir between the second surface of the support and the enhancer.

The injector may be made of silicon. The microneedle(s) may be made of silicon. The microneedle support may be made of silicon.

The or each microneedle may have a length such that, in use, it penetrates through the stratum corneum of the subject and does not penetrate to the dermis of the subject, so as to inject the substance into the epidermis of the subject. The microneedle, or each microneedle independently, may be between about 50 and about 150 microns long.

The at least one microneedle may be an array of microneedles. Each of said microneedles may be capable of penetrating to the epidermis of the subject so as to inject the substance into the epidermis. The array may comprise between about 500 and about 2000 microneedles.

In an embodiment there is provided an injection device for injecting a substance into a subject, said injection device comprising:
(i) an injector comprising a microneedle support and an array of microneedles, each extending from a first surface of said support, wherein each microneedle has a fluid channel extending through the microneedle and through the support, the fluid channel of each microneedle having an inlet aperture in a second surface of the support and an outlet aperture in the microneedle; and
(ii) an ultrasound generator for enhancing the rate of penetration of the substance into the subject.

In another embodiment there is provided an injection device for injecting a substance into a subject, said injection device comprising:
(i) an injector comprising a microneedle support and an array of microneedles, each extending from a first surface of said support, wherein each microneedle has a fluid channel extending through the microneedle and through the support, the fluid channel of each microneedle having an inlet aperture in a second surface of the support and an outlet aperture in the microneedle;
(ii) a reservoir for containing a fluid comprising the substance, said reservoir being disposed so as to allow the fluid to pass from the reservoir into the fluid channels through the inlet apertures thereof;
(iii) a piezoelectric crystal; and
(iv) a source of alternating current coupled to the piezoelectric crystal whereby the piezoelectric crystal is capable of generating ultrasound in response to an alternating current from said source.

In another embodiment there is provided an injection device for injecting a substance into a subject, said injection device comprising:
(i) an injector comprising a microneedle support and an array of microneedles, each extending from a first surface of said support, wherein each microneedle has a fluid channel extending through the microneedle and through the support, the fluid channel of each microneedle having an inlet aperture in a second surface of the support and an outlet aperture in the microneedle;
(ii) a reservoir for containing a fluid comprising the substance, said reservoir being disposed so as to allow the fluid to pass from the reservoir into the fluid channels through the inlet aperture thereof;
(iii) a piezoelectric crystal; and
(iv) a source of alternating current coupled to the piezoelectric crystal whereby the piezoelectric crystal is capable of generating ultrasound in response to an alternating current from said source;
   wherein each microneedle has a length such that, in use, it penetrates through the stratum corneum of the subject and does not penetrate to the dermis of the subject, so as to inject the substance into the epidermis of the subject.

In another embodiment there is provided an injection device for injecting a substance into a subject, said injection device comprising:
(i) an injector comprising a microneedle support and an array of microneedles, each extending from a first surface of said support, wherein each microneedle has a fluid channel extending through the microneedle and through the support, the fluid channel of each microneedle having an inlet aperture in a second surface of the support and an outlet aperture in the microneedle;
(ii) a piezoelectric crystal coupled to the microneedle support so as to define a reservoir between the second surface of the support and the crystal; and
(iii) a source of alternating current coupled to the piezoelectric crystal whereby the piezoelectric crystal is capable of generating ultrasound in response to an alternating current from said source;
   wherein each microneedle has a length such that, in use, it penetrates through the stratum corneum of the subject and does not penetrate to the dermis of the subject, so as to inject the substance into the epidermis of the subject.

In a second aspect of the invention there is provided a method for delivering a substance to a subject, said method comprising:
providing an injection device according to the first aspect;
applying the injector to the skin of the subject so that the at least one microneedle penetrates the skin of the subject;
supplying a fluid comprising the substance to the or each inlet aperture so as to allow said fluid to enter the fluid channel or channels; and
activating the enhancer so as to enhance the rate of penetration of the substance into the subject.

The following options may be used as part of the second aspect, and may be used independently or in any practical combination.

The enhancer may comprise an ultrasound generator. In this case the step of activating the enhancer may comprise causing the ultrasound generator to supply ultrasound to the skin of the subject adjacent to, or in the vicinity of, the microneedles.

The injection device may comprise a reservoir for containing the fluid comprising the substance. The reservoir may be disposed so as to allow the fluid to pass from the reservoir into the or each fluid channel through the inlet aperture thereof. In this case the step of supplying the fluid may comprise supplying the fluid to the reservoir.

The substance may be a therapeutic substance indicated for treating a condition of the subject. In this case the method may be a method for treating the condition of the subject and the step of supplying the fluid may comprise supplying a fluid containing a therapeutically effective dose of the substance to the aperture or apertures.

The step of applying the injector to the skin of the subject may be conducted so that the at least one microneedle penetrates through the stratum corneum of the subject to the epidermis. It may be conducted so that the at least one microneedle does not penetrate as far as the dermis of the subject. It may be conducted such that the subject does not feel pain. It may be conducted painlessly. It may be conducted such that the subject does not bleed in the vicinity of the applying.

In an embodiment there is provided a method for delivering a substance to a subject, said method comprising:
providing an injection device comprising (i) an injector comprising a microneedle support and an array of microneedles, each extending from a first surface of said support, wherein each microneedle has a fluid channel extending through the microneedle and through the support, the fluid channel of each microneedle having an inlet aperture in a second surface of the support and an outlet aperture in the microneedle; and (ii) an ultrasound generator for enhancing the rate of penetration of the substance into the subject;
applying the injector to the skin of the subject so that the microneedles penetrate the skin of the subject;
supplying the fluid comprising the substance to the inlet apertures so as to allow said fluid to enter the fluid channels; and
causing the ultrasound generator to supply ultrasound to the skin of the subject adjacent to the microneedles, thereby enhancing the rate of penetration of the substance into the subject.

In another embodiment there is provided a method for delivering a substance to a subject, said method comprising:
providing an injection device comprising (i) an injector comprising a microneedle support and an array of microneedles, each extending from a first surface of said support, wherein each microneedle has a fluid channel extending through the microneedle and through the support, the fluid channel of each microneedle having an inlet aperture in a second surface of the support and an outlet aperture in the microneedle; (ii) a reservoir for containing a fluid comprising the substance, said reservoir being disposed so as to allow the fluid to pass from the reservoir into the fluid channels through the inlet apertures thereof; and (iii) an ultrasound generator for enhancing the rate of penetration of the substance into the subject;
applying the injector to the skin of the subject so that the microneedles penetrate the skin of the subject;
supplying the fluid to the reservoir so as to allow said fluid to enter the fluid channels; and
causing the ultrasound generator to supply ultrasound to the skin of the subject adjacent to the microneedles, thereby enhancing the rate of penetration of the substance into the subject.

In another embodiment there is provided a method for treating a condition of a subject, said method comprising:
providing an injection device comprising (i) an injector comprising a microneedle support and an array of microneedles, each extending from a first surface of said support, wherein each microneedle has a fluid channel extending through the microneedle and through the support, the fluid channel of each microneedle having an inlet aperture in a second surface of the support and an outlet aperture in the microneedle; (ii) a reservoir for containing a fluid, said reservoir being disposed so as to allow the fluid to pass from the reservoir into the fluid channels through the inlet apertures thereof; and (iii) an ultrasound generator for enhancing the rate of penetration of the fluid into the subject;
applying the injector to the skin of the subject so that the microneedles penetrate the skin of the subject;
supplying a fluid containing a therapeutically effective dose of a therapeutic substance to the reservoir so as to allow said fluid to enter the fluid channels, said therapeutic substance being indicated for treating the condition of the subject; and
causing the ultrasound generator to supply ultrasound to the skin of the subject adjacent to the microneedles, thereby enhancing the rate of penetration of the fluid into the subject.

In a third aspect of the invention there is provided a process for making an injection device for injecting a substance into a subject, said process comprising coupling an injector to an enhancer for enhancing the rate of penetration of a substance into a subject, said injector comprising a microneedle support and at least one microneedle extending from a first surface of said support, wherein the or each microneedle has a fluid channel extending through the microneedle and through the support, the fluid channel of the or each microneedle having an inlet aperture in a second surface of the support and an outlet aperture in the microneedle.

The following options may be used as part of the third aspect, and may be used independently or in any practical combination.

The enhancer may be an ultrasound generator. The process may additionally comprise the step of coupling, e.g. electrically coupling, a source of alternating current to said ultrasound generator. The step of coupling the source to the generator may comprise attaching electrical connections to the ultrasound generator. The electrical connections may be electrically coupled to the source of alternating current.

The step of coupling the injector to the enhancer may comprise coupling the microneedle support to the enhancer so as to form a reservoir between the second surface of the support and the enhancer. The step of coupling the microneedle support to the enhancer may comprise coupling a spacer to the second surface of the support and coupling the enhancer to the spacer. The spacer may comprise glass. It may comprise, or be fabricated from, a glass wafer.

The process may additionally comprise the step of providing the injector. The process may comprise the step of fabricating the injector, or the microneedle support and/or the microneedle(s), from a silicon wafer using microfabrication techniques.

In an embodiment there is provided a process for making an injection device for injecting a substance into a subject, said process comprising:
coupling an injector to an ultrasound generator, said injector comprising a microneedle support and at least one microneedle extending from a first surface of said support, wherein the or each microneedle has a fluid channel extending through the microneedle and through the support, the fluid channel of the or each microneedle having an inlet aperture in a second surface of the support and an outlet aperture in the microneedle; and
coupling a source of alternating current to said ultrasound generator.

In another embodiment there is provided a process for making an injection device for injecting a substance into a subject, said process comprising:
providing an injector comprising a microneedle support and at least one microneedle extending from a first surface of said support, wherein the or each microneedle has a fluid channel extending through the microneedle and through the support, the fluid channel of the or each microneedle having an inlet aperture in a second surface of the support and an outlet aperture in the microneedle;
coupling the microneedle support to an ultrasound generator so as to form a reservoir between the second surface of the support and the ultrasound generator; and
coupling a source of alternating current to said ultrasound generator.

In another embodiment there is provided a process for making an injection device for injecting a substance into a subject, said process comprising:
fabricating an injector from a silicon wafer using microfabrication techniques, said injector comprising a microneedle support and at least one microneedle extending from a first surface of said support, wherein the or each microneedle has a fluid channel extending through the microneedle and through the support, the fluid channel of the or each microneedle having an inlet aperture in a second surface of the support and an outlet aperture in the microneedle;
coupling the microneedle support to an ultrasound generator so as to form a reservoir between the second surface of the support and the ultrasound generator; and
coupling a source of alternating current to said ultrasound generator.

The invention also provides an injection device made by the process of the third aspect of the invention. It also provides an injection device according to the invention when used for injecting a substance into a subject. It also provides the use of an injection device according to the invention for injecting a substance into a subject.

### BRIEF DESCRIPTION OF THE DRAWINGS

A preferred embodiment of the present invention will now be described, by way of an example only, with reference to the accompanying drawings wherein:

FIG. 1 is a diagram showing the reservoir in an injection device according to the invention;

FIG. 2 is a diagram showing penetration of microneedles into skin;

FIG. 3 is a diagram illustrating use of an ultrasound enhanced microneedle array for transdermal drug delivery;

FIG. 4 is a diagram illustrating the main steps of a process for fabricating microneedles: (a) deposition and patterning SiO₂ layer; (b) thermal oxidation of the walls of the fluid channels; (c) spray coating of photoresist and patterning of the SiO₂ layer; (d) deep RIE for the fabrication of the out-ring of microneedles; (e) deep RIE for fabrication of the reservoir; (f) deposition and patterning of the masking layers on glass substrate; (g) double-side wet etching of glass holes in HF; (h) anodic bonding of silicon microneedles chip and glass substrate; (i) bonding of PZT membrane to the glass substrate;

FIG. 5 is an electron micrograph of hollow silicon microneedles with slanted tips;

FIG. 6 is an electron micrograph of an array of silicon microneedles;

FIG. 7 is a photograph of an injection device according to the present invention;

FIG. 8 shows a diagram of a test device for determining penetration rates into skin; and

FIG. 9 is a graph showing penetration rates into skin under different experimental conditions.

### DETAILED DESCRIPTION OF THE INVENTION

The present invention provides an injection device for injecting a substance into a subject. The injection device comprises an injector and an enhancer. The injector comprises a microneedle support and at least one microneedle extending from a first surface of said support. The description herein refers primarily to the case in which there are more than one microneedle, however an injection device according to the invention may have a single microneedle, and the description, where appropriate, should be taken to include this case as well. Each microneedle has a fluid channel extending through the microneedle and through the support. The fluid channel may have a hydrophilic surface. The fluid contact surface of the fluid channel may be hydrophilic. The fluid channel may have hydroxyl groups, e.g. silanol groups, on the surface. Each fluid channel has in inlet aperture in (i.e. opening from) a second surface of the microneedle support and an outlet aperture in (i.e. opening from) the microneedle. Thus each fluid channel is such that a fluid can pass into the injector through the inlet aperture, pass through the fluid channel and pass out of the injector through the outlet aperture.

The enhancer is capable of enhancing the rate of penetration of the substance into the subject. Thus in operation of the injection device, the injector is used to introduce the substance into the subject, in particular to the skin of the subject, and the enhancer is used to enhance the rate of penetration of the substance into the subject, in particular into the skin of the subject. Thus in combination the injector and the enhancer provide an injection device which provides enhanced injection capabilities while avoiding or reducing or minimising discomfort to the subject.

The enhancer may be any type of enhancer capable of enhancing the rate of penetration of a substance into a subject, particularly into or through the skin of the subject. The enhancer may comprise an ultrasound generator, for example a piezoelectric material, e.g. a piezoelectric crystal. Suitable piezoelectric materials include naturally occurring crystals or other naturally occurring materials, man-made crystals, man-made ceramics and polymers. Suitable naturally occurring crystals include tourmaline, quartz, topaz, cane sugar, apatite and Rochelle salt (potassium sodium tartrate, KNaC₄H₄O₆.4H₂O). Other naturally occurring materials include bone. Suitable man-made crystals include berlinite (AlPO₄) and gallium orthophosphate (GaPO₄), which are quartz analogue crystals. Suitable man-made ceramics include the family of ceramics with perovskite or tungsten-bronze structures. These include barium titanate (BaTiO₃), lead zirconate titanate (Pb(ZrTi)O₃) (commonly known as PZT), strontium titanate (SrTiO₃) potassium niobate (KNbO₃), lithium niobate (LiNbO₃), lithium tantalate (LiTaO₃), bismuth ferrite (BiFeO₃), sodium tungstate (NaₓWO₃), Ba₂NaNb₅O₅ and Pb₂KNb₅O₁₅. A suitable polymer is polyvinylidene fluoride (PVDF). The piezoelectric crystal may comprise a PZT membrane.

The ultrasound generator may be capable of providing ultrasound at a frequency of between about 0.01 and about 10 MHz, or about 0.05 to 10, 0.1 to 10, 0.2 to 10, 0.5 to 10, 0.01 to 5, 0.01 to 2, 0.01 to 1, 0.01 to 0.5, 0.01 to 0.1, 0.01 to 0.05, 0.02 to 0.05, 0.1 to 5, 0.2 to 5, 0.05 to 2, 0.5 to 10, 1 to 10, 2 to 10, 5 to 10, 0.1 to 5, 0.1 to 2, 0.1 to 1, 0.1 to 0.5, 1 to 5, 2 to 5, or 0.5 to 2 MHz, e.g. about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 MHz. The ultrasound may be produced at a frequency of about 20 to about 50 kHz, or about 20 to 40, 20 to 30, 30 to 40, 40 to 50 or 30 to 40 kHz, e.g. about 20, 25, 30, 35, 40, 45 or 50 kHz. It may be capable of providing ultrasound with an intensity of between about 0.01 and 5 W/cm², or about 0.01 to 1, 0.01 to 0.5, 0.01 to 0.1, 0.01 to 0.05, 0.1 to 5, 0.5 to 5, 1 to 5, 0.1 to 1, 0.1 to 2, 0.1 to 2.5, 1 to 4, 1 to 3, 2 to 5, 3 to 4, 2 to 3 or 2 to 4 W/cm², e.g. about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5 or 5 W/cm². It may be capable of providing ultrasound to the fluid that is injected using the device. It may be capable of providing ultrasound to the skin of the subject into which the fluid is injected. It may be capable of providing ultrasound to the subject in the vicinity of the outlet aperture of the injector. It may be capable of providing ultrasound to the at least one microneedle of the device. It may be capable of providing ultrasound to more than one of these. The ultrasound may be transmitted to the subject through the fluid in the reservoir, through the fluid in the microneedles, through the microneedle support, through the microneedles, through the skin of the subject or through a combination of any two or more of these.

In the event that the enhancer comprises a piezoelectric crystal, the crystal may be coupled to a source of alternating current whereby the crystal is capable of generating ultrasound in response to an alternating current from said source. The crystal may be coupled to the source electrically so as to allow an alternating current from the source to be transmitted to the crystal so as to cause the crystal to generate ultrasound. The source of alternating current may be an AC generator. It may comprise a transformer. The source may be capable of generating an alternating current with a frequency of between about 0.01 and about 10 MHz, or about 0.02 to 0.05, or 0.02 to 0.04, 0.02 to 0.03, 0.03 to 0.04, 0.04 to 0.05, 0.03 to 0.05, 0.05 to 10, 0.1 to 10, 0.2 to 10, 0.5 to 10, 0.01 to 5, 0.01 to 2, 0.01 to 1, 0.01 to 0.5, 0.01 to 0.1, 0.01 to 0.05, 0.02 to 0.05, 0.1 to 5, 0.2 to 5, 0.05 to 2, 0.5 to 10, 1 to 10, 2 to 10, 5 to 10, 0.1 to 5, 0.1 to 2, 0.1 to 1, 0.1 to 0.5, 1 to 5, 2 to 5, or 0.5 to 2 MHz, e.g. about 0.01, 0.015, 0.02, 0.025, 0.03, 0.035, 0.04, 0.045, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 MHz. Commonly the frequency of the source will correspond to the frequency of the ultrasound generated by the piezoelectric crystal.

The piezoelectric crystal may be coupled to a source of a current that varies at a frequency such that the crystal generates the desired frequency of ultrasound. The frequency of variation may be as described above for alternating current. Commonly alternating current is described by the equation

v(t)=v(peak)*sin(ωt)

where v(t) is the time function of voltage, v(peak) is the peak voltage, ω is the angular frequency of the variation and t is the time variable. It will be understood that other regularly varying currents may also generate ultrasound when applied to a piezoelectric crystal. For example rectified alternating current may be applied wherein the above function applies when sin(ωt) is positive, and when sin(ωt) is negative v(t) is zero. Other alternatives include superposition of the above function on a constant offset voltage, so that v(t)=v₁*sin(ωt)+v₂, where v₁ and v₂ are constants, or v(t)=v₁*sin(ωt)+v₂(t), in which v₂(t) is a time dependent voltage function, e.g. a linearly increasing function. v₂(t) may vary on a different time scale to v(t), preferably at a substantially longer time scale. It will also be understood that in all of the above voltage functions, the variation may instead have a non-sinusoidal voltage variation, e.g. it may be a square wave, triangular wave or other time function of voltage, each with a frequency as described above for alternating currents.

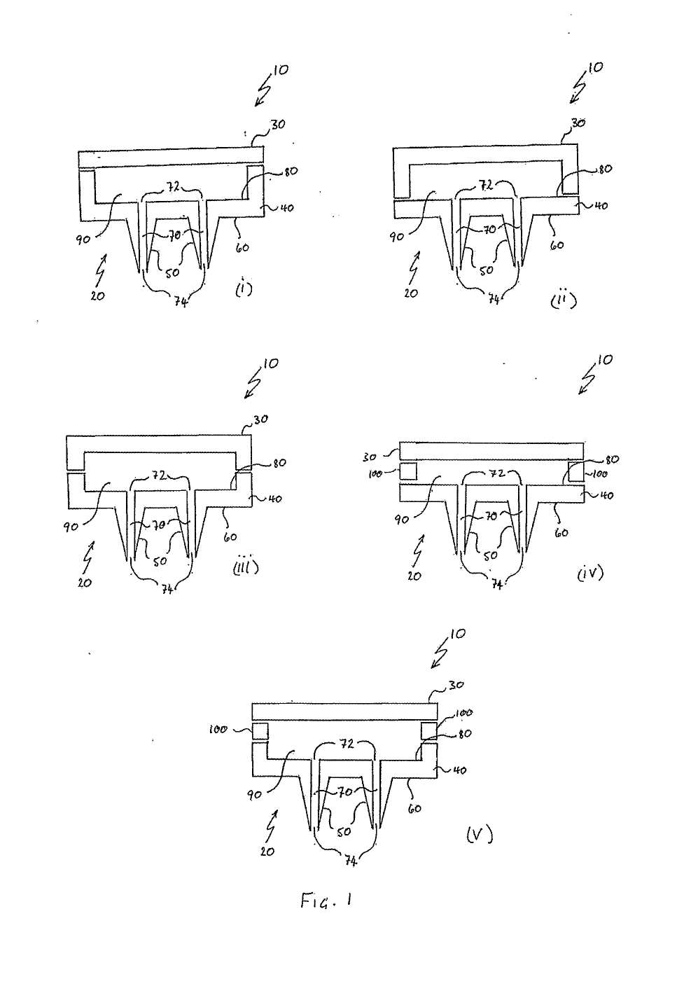

The injection device may additionally comprise a reservoir for containing the fluid comprising the substance. The reservoir may have a hydrophilic surface. It may have hydroxyl groups on the surface, particularly on the fluid contacting surface thereof. The reservoir may be in fluid communication with the fluid channel(s) of the injector. The inlet apertures of the fluid channels may open into the reservoir. In some embodiments, the enhancer is coupled to the microneedle support so as to define, or form, the reservoir between the second surface of the support and the enhancer. In such embodiments, either the enhancer or the support or both may have an indentation, or may have a suitable shape, such as a concave shape, so as to define, or form, the reservoir. Alternatively or additionally, the enhancer may be coupled to the microneedle support by means of a spacer, so that the spacer, the enhancer and the support define, or form, the reservoir. Some of these options are shown in FIG. 1. Thus in FIG. 1 injection device 10 comprises injector 20 and enhancer 30. Injector 20 comprises microneedle support 40 and microneedles 50 extending from first surface 60 of support 40. In FIG. 1, two microneedles 50 are shown for purposes of simplicity, however it will be understood that in many cases far more are present in practice. Each microneedle 50 has a fluid channel 70 therethrough. Each fluid channel 70 has an inlet aperture 72 in support 40 and an outlet aperture 74 in microneedle 50 near the tip thereof. Each fluid channel 70 passes through a microneedle 50 and through support 40 to second surface 80 of surface 40 so as to permit a fluid to pass from second surface 80 (in particular from inlet aperture 72) through fluid channel 70 and exit injector 20 through microneedle 50 (in particular through outlet aperture 74). Enhancer 30 is coupled to microneedle support 40 so as to form reservoir 90 between second surface 80 and enhancer 30. In diagrams i to iii of FIG. 1, enhancer 30 is coupled directly to microneedle support 40 so as to form reservoir 90. In diagrams iv and v, spacer 100 is present so that enhancer 30 is coupled to microneedle support 40 by means of spacer 100. In FIG. 1, spacer 100 is shown in two parts, since FIG. 1 shows a section through device 10. It will be understood that spacer 100 is in reality a continuos spacer between the perimeters of microneedle support 40 and enhancer 30. Thus in these diagrams, spacer 100, enhancer 30 and microneedle support 40 form reservoir 90. In diagrams i, iii and v, support 40 is shown as concave, and in diagrams ii and iii, enhancer 30 is shown as concave. In diagram iv, neither enhancer 30 nor support 40 is concave, both being flat, or planar, and the presence of spacer 100 is necessary to form reservoir 90. Clearly the volume of reservoir 90 may be adjusted by adjusting the depth of the concavity of either enhancer 30 or support 40 or both, or, if present, the depth of spacer 100. The reservoir, if present, may comprise an inlet port (not shown in FIG. 1), which may be resealable, so that the reservoir may be refilled following or during use of the injection device and consequent injection of fluid from the reservoir into a subject.

It should be noted that in diagrams i, iii and iv of FIG. 1, in which the microneedle support is not planar, the “second surface” of the microneedle support (described in this specification) may include not only that area in which inlet apertures 72 are located, but also that area to which enhancer 30 is coupled (either directly, in the case of i and iii or indirectly in the case of v).

In operation a fluid comprising the substance to be injected into the subject is located in reservoir 90. Microneedles 50 are inserted into the skin of the subject so as to penetrate through the stratum corneum and into the epidermis. Fluid can diffuse from the reservoir and through channels 70 into the epidermis of the subject. Activation of enhancer 30 enhances penetration of the fluid into the subject.

The injector, or portions thereof, may be made of a metalloid, for example silicon, a metal, for example titanium or stainless steel, or a plastic. The injector may be made of silicon. The microneedles may be made of silicon or may be primarily made of silicon. The microneedle support may be made of silicon or may be primarily made of silicon. Fabrication from silicon enables well-known microfabrication processes to be used in making the injection device. The silicon may be “p” type silicon. The resistivity of the silicon may be between about 1 and about 20 Ωcm, or about 1 to 10, 1 to 5, 5 to 20, 10 to 20, 5 to 15, 5 to 10 or 10 to 15 Ωcm, e.g. about 1, 2, 3, 4, 5, 6, 7, 8, 9, 10, 11, 12, 13, 14, 15, 16, 17, 18, 19 or 20 Ωcm.

The microneedle support may have a thickness of about 50 and about 500 microns, or about 50 to 250, 50 to 200, 50 to 150, 50 to 100, 100 to 500, 250 to 500, 100 to 250, 150 to 250 or 100 to 200 microns, e.g. about 50, 100, 150, 200, 250, 300, 350, 400, 450 or 500 microns. It may be substantially planar. The portion of the microneedle support from which the microneedles extend may be substantially planar. The first surface (from which the microneedles extend) and the second surface (onto which the inlet apertures open) may be opposite surfaces. They may be substantially parallel to each other.

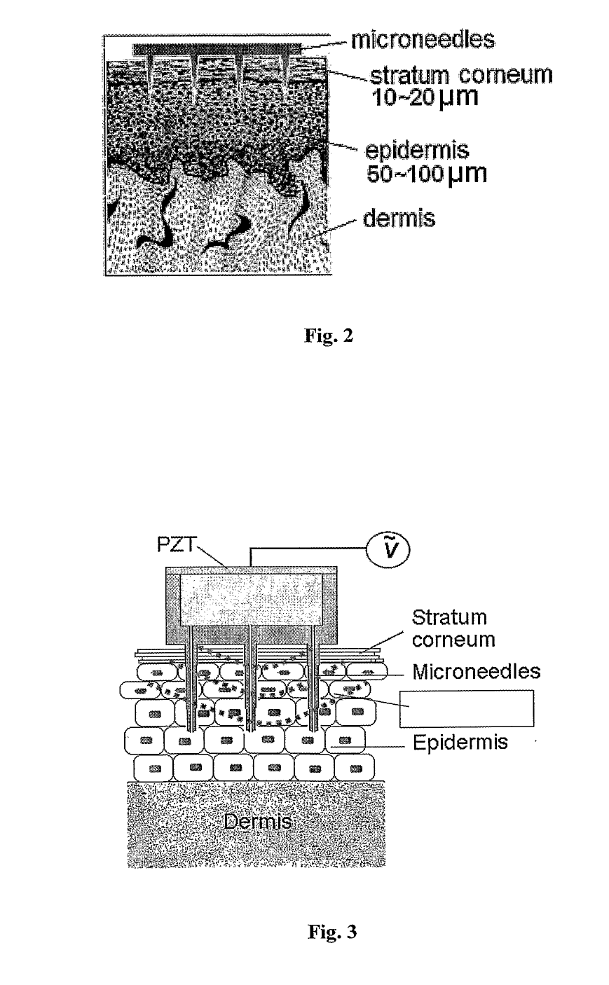

The microneedles may be sufficiently long that, in use, they penetrate through the stratum corneum of the subject to the epidermis and do not penetrate to the dermis of the subject, so as to inject the substance into the epidermis of the subject. This is illustrated in FIG. 2. It is useful for the microneedles to penetrate through the stratum corneum in order to pass this barrier to absorption of the substance. It is however useful for the microneedles not to penetrate as far as the dermis, so as to avoid discomfort and/or pain and/or bleeding associated with deeper injection. Thus for use with a human subject the microneedle, or each microneedle independently, may be between about 50 and about 150 microns long. They may be about 50 to 100, 100 to 150, 70 to 120, 70 to 100 or 100 to 130 microns long, e.g. about 50, 60, 70, 80, 90, 100, 110, 120, 130, 140 or 150 microns long. It will be understood that the length may differ from this when the injection device is designed for use with non-human subjects, particularly those with skins having layers of different thicknesses to those of humans. The outside diameter of the microneedles may be between about 20 and about 100 microns, or about 20 to 80, 20 to 50, 30 to 100, 50 to 100, 70 to 100, 50 to 70 or 30 to 60 microns, e.g. about 20, 25, 30, 35, 40, 45, 50, 55, 60, 65, 70, 75, 80, 85, 90, 95 or 100 microns. The microneedles may be any shape suitable for insertion into the skin of a subject. In the event that the shape of the microneedles does not have a constant diameter, the above outside diameter values may be the mean or the maximum diameter. They may be for example cylindrical, cylindrical with a conical, hemispherical or pyramidal end. They may be elongate with a polygonal cross-section (e.g. triangular, square, pentagonal, hexagonal, octagonal, decagonal, dodecagonal etc., these being either regular or irregular polygons), pyramidal (having a regular or irregular polygonal base having e.g. 3, 4, 5, 6, 7, 8, 9, 10, 11, 12 or more than 12 sides), or they may be some other suitable shape. They may be acicular.

The diameter of the fluid channels within the microneedles may be between about 10 and about 50 microns (provided that it is smaller than the outside diameter of the microneedle), or about 10 to 40, 10 to 30, 20 to 50, 20 to 50 or 15 to 25 microns, e.g. about 10, 15, 20, 25, 30, 35, 40, 45 or 50 microns. The wall thickness of the microneedles may be between about 5 and about 20 microns, e.g. about 5 to 15, 5 to 10, 10 to 20 or 15 to 20 microns, e.g. about 5, 10, 15 or 20 microns. The wall thickness may vary, in that the channels may not be centrally located within the microneedles, but may be located eccentrically so as to produce a slanted tip to the microneedle. The channels may have a round cross-section, or it may have a regular or irregular polygonal cross-section having for example 3, 4, 5, 6, 7, 8, 9, 10, 11, 12 or more than 12 sides, or may be some other shape (e.g. oval, elliptical etc.). The outlet aperture of the fluid channel may be in the tip of the microneedle, or may be elsewhere in the microneedle. It is preferably sufficiently close to the tip of the microneedle that, when inserted into the skin of a subject, the aperture is located in the epidermis of the subject so as to enable a fluid to be injected through the microneedle into the epidermis. Thus in an injection device for use with a human subject, the distance from the first surface of the microneedle support and the outlet aperture may be between about 20 and about 150 microns, or about 20 to 120, 20 to 100, 20 to 80, 20 to 50, 30 to 150, 50 to 150, 100 to 150, 30 to 120, 30 to 100, 30 to 50 or 50 to 100 microns, e.g. about 20, 25, 30, 35, 40, 45, 50, 60, 70, 80, 90, 100, 110, 120, 130, 14 or 150 microns.

In some embodiments, the injector has a plurality of microneedles. These may be arranged in array, e.g. a regular array. In such embodiments, each of the microneedles should be capable of penetrating to the epidermis of the subject so as to inject the substance into the epidermis, as described above. The array may comprise between about 500 and about 2000 microneedles, or may comprise more or less than this range. The number of microneedles will depend on such factors as the diameter of the channels, the desired delivery rate of the fluid, the concentration of active in the fluid etc. The array may for example comprise about 500 to 1500, 500 to 1000, 1000 to 2000, 700 to 1200 or 700 to 1000 microneedles, e.g. about 500, 600, 700, 800, 900, 1000, 1100, 1200, 1300, 1400, 1500, 1600, 1700, 1800, 1900 or 2000 microneedles. The array may be a square array, i.e. it may have the same number of microneedles on each side, or it may be a rectangular array, or it may be a circular array (in which the microneedles are arranged in concentric circles), or it may be a spiral array, or it may be some other design of array. In the case of a square array, each side of the array may have between about 20 and 40 microneedles, or between about 20 and 30, 30 and 40 or 25 and 35 microneedles, e.g. about 20, 25, 30, 35 or 40 microneedles. In the case of a rectangular array, each side of the array may, independently, be as described above. The distance between microneedles (e.g. between the centre points of the microneedles, or between the points where the needles meet the support) in an array may be between about 100 and about 500 microns, or about 100 to 400, 100 to 300, 200 to 500, 300 to 500, 200 to 400, 200 to 300, 300 to 400 or 250 to 350 microns, e.g. about 100, 150, 200, 250, 300, 350, 400, 450 or 500 microns. The length of the side of a rectangular or square array, or of the diameter of a round or elliptical (i.e. major or minor axis) may independently be between about 5 and 20 mm, or between about 5 and 10, 10 and 20 or 10 and 15 mm, e.g. about 5, 6, 7, 8, 9, 10, 11, 12, 13, 14, 15, 16, 17, 18, 19 or 20 mm.

The invention also provides a method for delivering a substance to a subject. In this method, an injection device according to the invention is provided, and the injector of the device is applied to the skin of the subject so that the at least one microneedle penetrates the skin of the subject. A fluid (commonly a liquid) comprising the substance is supplied to the second surface of the microneedle support so as to allow the fluid to enter the fluid channel(s) of the device through the inlet aperture(s). The enhancer is activated so as to enhance the rate of penetration of the substance into the subject.

The nature of the enhancer will control the nature of the activation. Thus, if the enhancer comprises an ultrasound generator, e.g. a piezoelectric crystal, the step of activating the enhancer may comprise causing the ultrasound generator to supply ultrasound to the skin of the subject adjacent to the microneedles. This may comprise supplying the ultrasound generator with an AC (alternating current) current as described earlier. The ultrasound generator should supply ultrasound to the region of the subject's skin adjacent to the microneedle(s), in particular adjacent the location where the channel exits the microneedle(s). The ultrasound may be transmitted to that region of the subject's skin by transmittal through the fluid in the reservoir (if present) or through the injector, or through both.

The steps of applying the injector to the skin, supplying the fluid to the microneedle support (in particular to the second surface thereof, i.e. to the inlet apertures) and activating the enhancer may be conducted in any desired order. In some embodiments, the fluid will be located in the reservoir, which is in contact with the second surface of the microneedle support, and the injector will be then applied to the skin after which the enhancer may be activated in order to increase the rate of delivery of the fluid to the subject. It is of course generally desirable that the activation of the enhancer continue for the period over which injection of the fluid into the subject occurs, so that for that period the rate is enhanced. In some cases however the activation may be switched on and off as required in order to vary the rate of delivery of the substance to the patient. In other embodiments the injector will be applied to the skin of the subject, the activation of the enhancer will be commenced, and thereafter the fluid will be applied to the second surface of the microneedle support. As noted above, other orders of these steps are envisaged by the present invention.

As noted above, the injection device may comprise a reservoir for containing the fluid comprising the substance, said reservoir being disposed so as to allow the fluid to pass from the reservoir into the fluid channels through the inlet apertures thereof. In this event the step of supplying the fluid may comprise supplying the fluid to the reservoir. If no reservoir is present, the step of supplying the fluid may comprise supplying the fluid to the second surface of the microneedle support, in particular to the inlet apertures of the fluid channels. The reservoir, or the channels, may be supplied with fluid from a tube or other conduit suitable for conveying the liquid to the reservoir or channels. In some embodiments, a reservoir is in fluid communication with the channels by means of a tube or other suitable conduit. In these embodiments, fluid from the reservoir passes through the conduit to the channels, and then passes through the channels to the subject to be injected. The supplying may comprise applying a pressure to the fluid to cause it to pass through the fluid channels to the subject, or it may not comprise applying pressure to the fluid. In the latter case, the fluid may pass into the subject by diffusion from the channels. The rate of this diffusion may be enhanced by activation of the enhancer, for example by activating an ultrasound generator so as to cause it to generate ultrasound.

The substance may be a therapeutic substance indicated for treating a condition of the subject. It may be a drug. It may be a vaccine. It may be a protein. It may be an enzyme. It may be a peptide, e.g. a polypeptide or an oligopeptide. It may be a saccharide, e.g. a polysaccharide. It may be an antibody or an antibody fragment. It may be a mixture of any two or more of these. It may be a macromolecular or high molecular weight substance or it may be a low molecular weight substance. It may comprise a variety of molecular weights. If the substance is a therapeutic substance, the method may be a method for treating a condition of the subject and the step of supplying the fluid may comprise supplying a fluid containing a therapeutically effective dose of the substance to the second surface of the microneedle support. The step of supplying the fluid may comprise supplying the fluid at a therapeutic rate for the substance. The therapeutically effective dose may be delivered over a sufficient time, or at a sufficient rate, that toxic or otherwise undesirable levels of the substance are not generated in the subject. This will depend on the toxicity and therapeutic efficacy of the substance and the nature and size of the subject. The subject may be a vertebrate. The vertebrate may be a mammal, a marsupial or a reptile. The mammal may be a primate or non-human primate or other non-human mammal. The mammal may be selected from the group consisting of human, non-human primate, equine, murine, bovine, leporine, ovine, caprine, feline and canine. The mammal may be selected from a human, horse, cattle, cow, ox, buffalo, sheep, dog, cat, goat, llama, rabbit, ape, monkey and a camel, for example. The subject may be a domesticated animal. It may be a pet. It may be a farm animal.

The substance may be in solution in the fluid, or it may be in suspension, or it may be emulsified, or it may be in a microemulsion, or it may be dispersed in the fluid, or it may be some combination of these (e.g. it may be partly in solution and partly emulsified). In the event that the substance is not in solution, the particle or droplet size of the substance should be smaller than the minimum diameter of the fluid channels of the injection device. Thus the fluid may be a solution, or it may be an emulsion, or it may be a microemulsion, or it may be a suspension, or it may be a dispersion, or it may be more than one of these. The fluid may be polar. It may be aqueous. It may comprise a solvent that is miscible with water, e.g. a lower alcohol such as ethanol or isopropanol.

The device of the present invention may be made by coupling an injector to an enhancer, these being as previously described. In the event that the enhancer is an ultrasound generator, and the process may additionally comprise the step of coupling a source of alternating current to said ultrasound generator. Thus for example the process may comprise coupling a piezoelectric crystal to the injector, and coupling a source of alternating current to the crystal (either before or after coupling it to the injector). The source of alternating current may be connected by means of electrically conducting wires, optionally using terminals which may be affixed to the crystal.

The step of coupling the injector to the enhancer may comprise coupling the microneedle support to the enhancer so as to form a reservoir between the second surface of the microneedle support and the enhancer. This has been described earlier with reference to FIG. 1. The microneedle support may be coupled to the enhancer by means of an intermediate substance. The intermediate substance may be for example a glue or adhesive or solder, or it may be a spacer (e.g. a glass, ceramic, polymeric or other type of spacer), or may be some combination of these. The intermediate substance may serve to couple the support to the enhancer, and in some embodiments may serve to partially form the reservoir.

The process may additionally comprise the step of providing the injector. The step of providing the injector may comprise fabricating the injector. Then nature of this step will depend in part on the nature of the injector, the design of the injector, the material(s) from which the injector is made etc. The injector may be made from silicon. It may be made from a metal (e.g. steel, stainless steel, titanium, gold, platinum, palladium), a ceramic (silica, alumina, titania, zirconia, a mixed oxide ceramic comprising two or more of silicon, aluminium, titanium and zirconium) or some other suitable material. The fabrication of the injector may comprise moulding, etching, forming or some other process, or may comprise a combination of these. The process may for example comprise the step of fabricating the injector, or the microneedle support and/or the microneedle(s), from a silicon wafer using microfabrication techniques. The process may comprise the step of rendering hydrophilic at least one of the contact surfaces of the injector, e.g. the fluid contact surfaces of the fluid channels and/or the fluid contact surface of the reservoir.

### DETAILED DESCRIPTION OF THE PREFERRED EMBODIMENTS

A preferred embodiment of the present invention provides an ultrasonic enhanced microneedle array for transdermal drug delivery. Thus in an embodiment of the invention a hollow microneedle array has been combined with an ultrasonic emitter in order to enhance the diffusion of various drugs and/or biological compounds into the skin of a subject. This combines the advantages conferred by microneedles for transdermal drug delivery with the advantages of ultrasonic delivery of drugs.

Microneedle devices have been developed for controlled transdermal drug or biological fluid delivery in a minimum invasion, painless, and convenient manner. The present inventors have combined a PZT membrane (in the device as ultrasonic emitter) with hollow microneedles, and employed ultrasonic energy to enhance the diffusion rate of a substance to be delivered by the device. This enables the delivery of sophisticated and large molecular species or macromolecular compounds into the skin. Combined with the advantages of microneedles for the transdermal drug delivery, the PZT ultrasonic emitter provides continuous ultrasonic energy to the fluid media, and improves the diffusion rate into the skin. It also helps large molecular compounds to readily diffuse into the skin, and reduces the risk of clogging of the microneedles.

Various types of microneedle have been developed for transdermal drug delivery, however, the rate of delivery has hitherto been limited by passive diffusion from the microneedles into the subject. Furthermore, the molecular size of the drugs or therapeutic compounds that may be delivered using microneedles has been constrained by passive diffusion. One means to overcome the problems associated with the diffusion phenomenon is to provide more “energy” to drug molecules or other substances to be delivered by the injection device. This “energy” may be for example generated by an ultrasonic enhancer. Ultrasonic energy is known to improve the rate and molecular size of diffusion into the skin.

The preferred range of ultrasound frequencies for medical diagnostic purposes is usually between 0.5 MHz and 5 MHz and the preferred range of intensities is between 2 and 4 W/cm². For the present invention, however, more common ranges are about 20 to about 50 kHz and about 0.1 to about 2.5 W/cm². These ranges are variable according to the species of subject, nature of the substance to be delivered and site of infusion, and values outside these ranges may be used after testing to determine optimum parameters to achieve the desired levels while minimizing damage to the infusion site.

Ultrasound energy can be used to enhance the skin diffusion and penetration of active substances. The inventors hypothesise as follows regarding this enhancement. When the skin is exposed to ultrasound, the waves propagate to a certain level and may cause several effects that assist the fluid diffusion. One of these effects is the formation and subsequent collapse of gas bubbles in a liquid, which is called cavitation. The force of cavitation is thought to cause the formation of holes in the keratinocytes, enlarging of intercellular spaces, and perturbation of stratum corneum lipids and epidermis tissue. It is hypothesized that oscillations of the cavitation bubbles induce disorder in the lipid bilayers, thereby enhancing transdermal transport. Another possible effect is heating, which is mainly due to the energy loss of the propagating ultrasound wave due to scattering and absorption effects. The resulting temperature elevation of the skin is typically in the range of several degrees centigrade (e.g. between about 1 and about 5° C., or about 1 to 3, 2 to 5 or 2 to 4° C., e.g. about 1, 1.5, 2, 2.5, 3, 3.5, 4, 4.5 or 5, or possibly more than 5° C.). This temperature rise may increase the fluidity of the stratum corneum and epidermis, as well directly increase the diffusivity of molecules through the skin barrier. These main effects may be assisted by acoustic microstreaming caused by the acoustic shear stress which is due to unequal distribution of pressure forces. In addition, ultrasound can push particles through by means of a pressure increase in the epidermis.

Ultrasonic energy is a potential method to improve the rate and molecular size of diffusion into the skin. In the present invention, the inventors have combined a microneedle array with an ultrasonic emitter, so as to enhance the diffusion of various drugs and biological compounds into the skin, with all the advantages of microneedles for the transdermal drug delivery.

Standard microfabrication techniques may be used for the fabrication of silicon microneedles arrays according to the invention. FIG. 2 shows a schematic view of transdermal drug delivery with microneedles. Thus the skin commonly comprises an outer layer, or stratum corneum, an epidermis adjacent the stratum corneum, and a dermis adjacent the epidermis. The dermis comprises nerve cells and blood vessels. As shown in FIG. 2, when the microneedles of the invention are inserted into the skin, they preferably penetrate through the stratum corneum and into the epidermis, but do not reach the dermis. Microneedle arrays may be inserted into the skin and create conduits for transport across the stratum corneum. Once a drug or compound crosses the stratum corneum, it can diffuse through the deeper tissue and be taken up by the underlying capillaries for systemic administration. In addition, due to their lengths of around 100 μm, microneedle arrays can pierce skin painlessly since they do not stimulate nerves in the deeper dermis. Thus microneedles can create pathways into the skin for drug delivery and may be painless due to their size.

A suitable device according to the present invention comprises two major parts: a hollow microneedle array and an ultrasonic emitter. Bulk micromachining technologies have been used to fabricate the out-of-plane hollow silicon microneedle array which provides a high permeability through the stratum corneum of skin and causes minimum invasion and pain. The array provides a large injection volume capacity and good uniformity of injection of drugs into the skin tissue. The silicon microneedle array is then bonded to another piece of glass substrate, which has a cavity to act as a reservoir and is then attached with to commercial PZT membrane. The PZT membrane may be excited with an AC generator with frequency of 20 kHz, thereby stimulating it to act as an ultrasonic emitter. An effective ultrasound frequency for transdermal delivery according to the present invention is in the frequency range of about 20 to about 50 kHz, or about 20 to about 30 kHz. This is different from the ultrasound frequency commonly for usual medical diagnostic or therapeutic purposes, which is between 0.5 MHz and 5 MHz. These frequencies appear to be suitable since the gaseous cavitation effect is not as readily generated using higher frequency ultrasound.

Similarly, a suitable power density for transdermal drug delivery may also be different from that used in usual medical therapeutic ultrasound. A suitable power density for transdermal delivery is between about 0.1 W/cm² and about 2.5 W/cm².

Advantages of the device are:
a microneedle array in combination with ultrasonic emitter has good structural strength, high permeability and low flow resistance of fluids into the skin;
the array of 30 by 30 microneedles provides for a large and uniform area of drug diffusion to the tissue
the PZT ultrasonic emitter provides continuous ultrasonic energy to the fluid, and thereby inducing the epidermis to act as a porous structure, and assisting the large molecular compounds to diffuse more readily into the skin, and
lowered the risk for clogging of the microneedles.

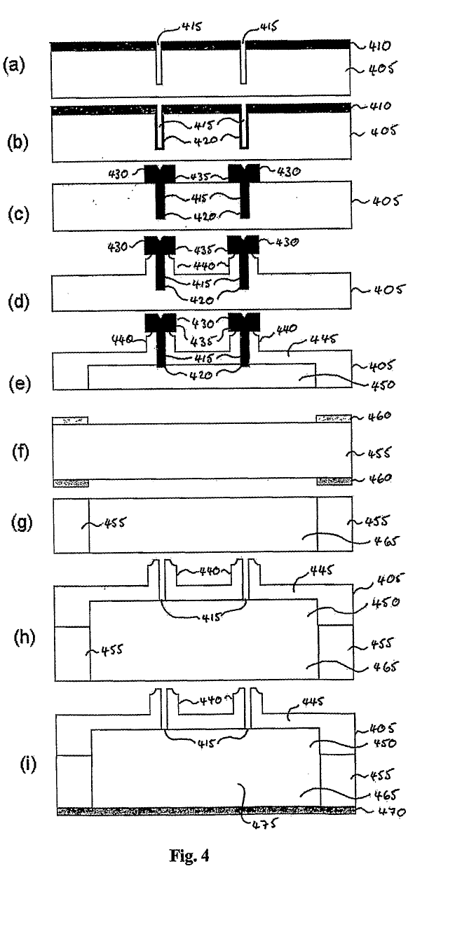

FIG. 3 shows a combination of ultrasound and microneedle array for the transdermal drug delivery. Thus an alternating current source V is shown connected to a PZT ultrasonic source which, together with the microneedles, forms a reservoir. As in FIG. 2, the microneedles are shown penetrating through the stratum corneum to the epidermis, but not reaching the dermis. FIG. 3 shows ultrasound, originating from the PZT crystal, penetrating through the skin of the subject. The combination of microneedles with an ultrasonic enhancer is a novel approach for transdermal delivery, and provides the improvement of facilitating transdermal drug transport with active diffusion. The chip consists of two parts: a hollow microneedle array and an ultrasound emitter. The hollow silicon microneedle array is fabricated with typical micromachining technologies, and then bonded to another substrate (glass) with PZT membrane. The microneedles have the length of 100 μm, out-diameter of 80 μm, inner-diameter of 40 μm, and array in the number of 30 by 30. The glass substrate is fabricated with a cavity, and commercial variable PZT membrane is attached to form a reservoir. In use of the microneedles, the primary skin barrier of stratum corneum is penetrated and the drug is delivered into the epidermis layer without pain and bleeding. At the same time, the PZT membrane is excited with high frequency AC voltage, and functions as an ultrasound emitter. The ultrasound is emitted to the skin tissue, and is thought to cause cavitations in the epidermis, generate temperature rise by energy loss of ultrasound propagation, and thereby enhance transdermal drug transport.

Advantages of ultrasound enhanced microneedles according to the present invention are enhancement of large-size molecules transdermal drug delivery, and/or high rate and/or quantity and/or uniformity of the active drug diffusion.

### Example 1

### Device Fabrication

The ultrasonic enhanced microneedle array device was fabricated with two wafers (one silicon wafer and the other one glass wafer) and then packaged with PZT thick membrane. The main steps of the fabrication process are presented in FIG. 4.

A 4″ silicon wafer 500 μm-thick, “p” type, with a resistivity between 1-20 Ωcm, was used for fabrication of the hollow microneedles array. The fabrication sequence consisted of etching of the inner holes or channels, secondly processing of an outer ring, and finally etching of the backside reservoir. The wafer was cleaned in piranha solution (H₂SO₄/H₂O₂ in ratio of 2/1) at 120° C. for 20 minutes, rinsed in deionised water and spun-dried.

A SiO₂ mask was generated for the fabrication of the holes (fluid channels) of the microneedles. A 2 μm-thick SiO₂ layer was grown in a Tystar furnace. A photoresist mask (AZ7220 from Clariant) was used to pattern the SiO₂ layer in an RIE (reactive ion etching) system using CF₄/O₂ gas mixture. Using SiO₂ as masking layer 250 μm-deep holes were performed in the silicon wafer using a classical Bosch process (SF₆/C₄F₈) on a Deep RIE system (Adixen AMS 100 Si) (FIG. 4a). Thus FIG. 4a shows silicon wafer 405, with silica layer 410. Holes 415 penetrate through silica layer 410 and partially through silicon wafer 405.

The passivation layer, resulting from the Bosch process, was removed using an annealing at 600° C. in vacuum. A second thermal oxidation, 1 μm thick, was performed in order to protect the shape of the holes during the next RIE fabrication steps (FIG. 4b).

Thus in FIG. 4b, oxidation layer 420 is shown on the insides of holes 415. The second photoresist mask, was applied using a spray coating process (EVG 101 system). After developing the layer, a hard baking process was performed in an oven for slow removal of the solvent from the photoresist mask. The patterning of the SiO₂ layer was performed using a similar process to that described above (using CF₄/O₂ gas mixture with RIE equipment)—FIG. 4c. Thus FIG. 4c shows the remaining portions 430 of the photoresist mask, which penetrate into holes 415, with residual portions 435 of the original silica layer.

The external shape of the silicon microneedles was define using an isotropic process in a deep RIE system followed by an anisotropic process (Bosch—previous described) (FIG. 4d). Thus FIG. 4d shows microneedles 440, having holes 415 lined with layer 420 and having photoresist 430 therein.

Finally, a third anisotropic Bosch process was performed through a SiO₂ mask from the back of the wafer (FIG. 4e). Thus FIG. 4e shows microneedles 440, having holes 415 lined with layer 420 and having photoresist 430 therein. In FIG. 4e, silicon wafer 405 has been formed into a shape having support 445 and cavity 450. Holes 415 penetrate through support 445 to cavity 450. The photoresist mask was removed in a classical photoresist stripper, while the oxide mask was removed in BOE (buffer oxide etcher). A dry oxidation (100 nm-thick) was performed in order to achieve a hydrophilic surface of the microneedles holes surface.

The glass substrate was fabricated and bonded to the silicon wafer to form the reservoir (in order to increase the reservoir volume and to permit a connection with a syringe needle). Two masking layers composed of amorphous Si/SiC/Photoresist were deposited on the both sides of an 1 mm-thick glass wafer (Corning 7740, Pyrex®)—FIG. 4f. Thus FIG. 4f shows glass wafer 455, together with masking layer 460, portions of which have been removed to expose wafer 455. The layers were deposited in a PECVD (plasma enhanced chemical vapour deposition) reactor, while the etching through the photoresist mask was performed in an RIE system using SF₆. Using these masking layers the glass wafer was etched-through in highly-concentrated HF solution (49%)—FIG. 4g. Thus FIG. 4g shows wafer with cavity 465 therein. Masking layer 460 has been removed in FIG. 4g.

After the removing the masking layer —FIG. 4g—using same RIE process as was used for patterning, the glass wafer was anodically bonded on the silicon wafer with microneedles —FIG. 4h. Thus FIG. 4h shows silicon wafer 405, having needles 440 with holes 415 therethrough, and cavity 450. Wafer 405 is bonded to glass wafer 455 having cavity 465 therein. Finally the wafer was diced into silicon-on-glass (SOG) chips of 12 mm by 12 mm square. On the SOG chips a commercially available thick PZT membrane was bonded using SnAu ball-soldering —FIG. 4i. Thus FIG. 4i shows silicon wafer 405, having needles 440 with holes 415 therethrough, and cavity 450. Wafer 405 is bonded to glass wafer 455 having cavity 465 therein. Wafer 455 is bonded to PZT membrane 470 so as to form reservoir 475, which is defined by membrane 470 and wafers 455 and 405, and comprises cavities 450 and 465.

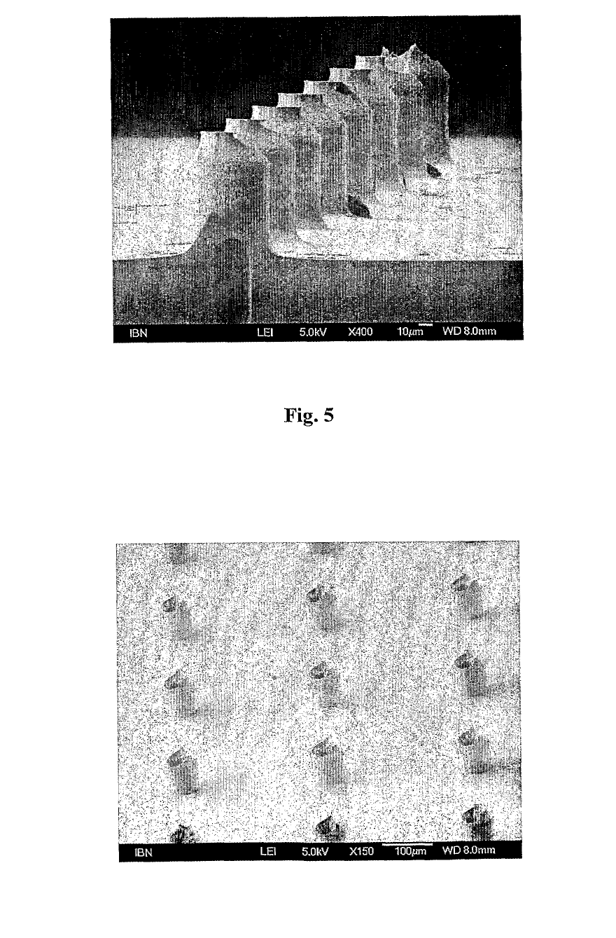

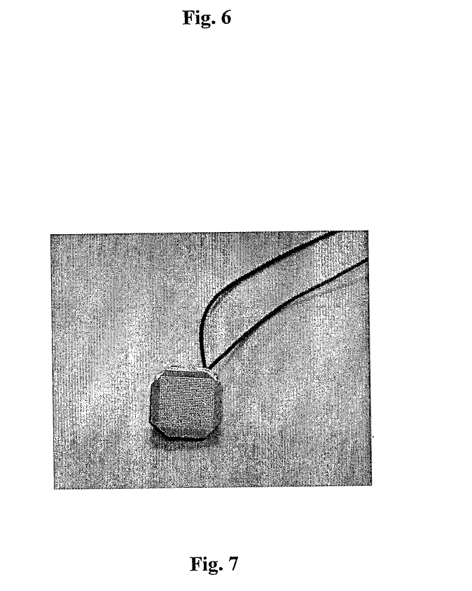

FIG. 5 and FIG. 6 show the fabrication results of the hollow microneedle array, with microneedles of length of 100 μm, inner-diameter of 40 μm and outer-diameter of 80 μm. The inner-holes can be designed to be eccentric to the outer-ring, so as to generate slanted tips on the microneedles, and facilitate the penetration into the skin.

Thus in the present invention an ultrasonic enhanced microneedle array has been developed, and may be used for transdermal drug delivery. With the combination of the microneedles and ultrasound, the rate of transdermal drug transport may be greatly enhanced. Furthermore, large-sized molecules such as vaccines, complicated bio-agents and macro-compounds can be also delivered into the body transdermal, with high permeability and no pain.

### Example 2

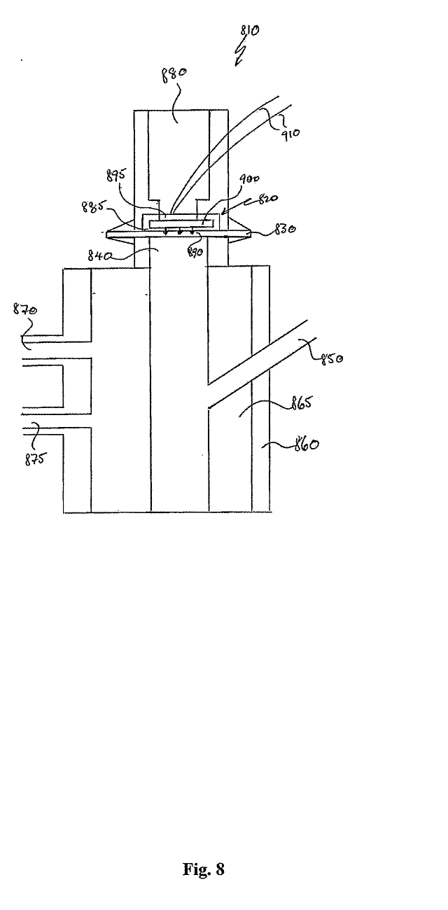

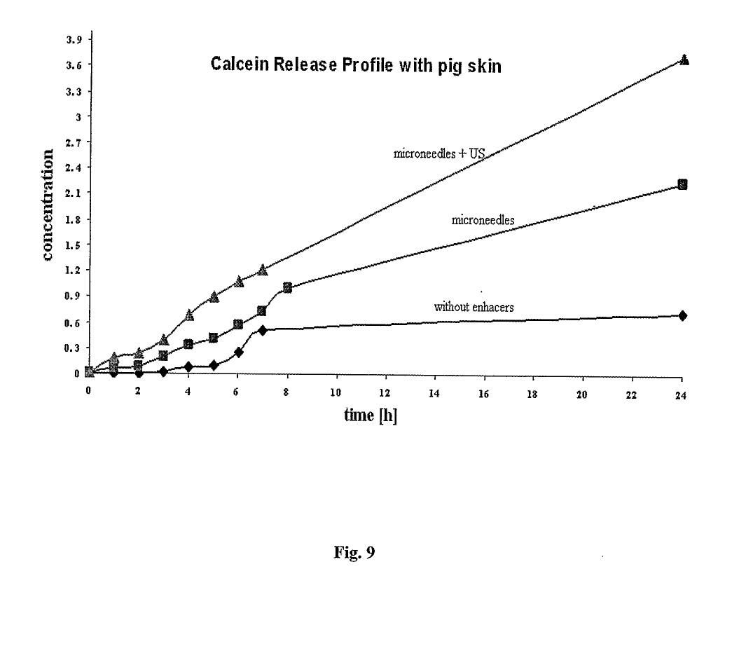

An injection device according to the present invention was constructed as described above. A photograph of the device is shown in FIG. 7. Testing was performed using calcein on pig skin, using the apparatus in FIG. 8. The graph of FIG. 9 presents the results of testing in three different situations: without enhancers, with hollow microneedles and using the method of the present invention, using microneedles with ultrasonic enhancement. With reference to FIG. 8, test device 10 comprises injection device 20 which has been applied to skin sample 30. Pig skin sample 30 communicates with a receiving liquid in receiving chamber 40. Arm 50 is provided to chamber 40 for inserting or withdrawing the receiving liquid. Chamber 40 is located in Franz cell 60, which comprises a water bath 65 and inlet and outlet ports 70 and 75 respectively for maintaining the receiving liquid at a constant temperature. Sample chamber 80 is provided for holding a test sample. Injection device 20 comprises an injector comprising microneedle support 85 having microneedle array 90 extending downwards therefrom, as described elsewhere in this specification. Device 20 also comprises PZT crystal 95 for enhancing the rate of penetration of the test sample through skin sample 30. PZT crystal adjoins reservoir 100 of device 20, which communicates with sample chamber 80. PZT crystal 90 has leads 110 attached so as to supply alternating current to crystal 90.

In order to conduct the test using test device 10, calcein solution (1 mmol, 0.523 mg/ml) was placed in chamber 80 so as to supply the solution to device 20, and a receiving liquid (PBS: phosphate buffered saline) was loaded into chamber 40. The receiving liquid was maintained at 37° C. through the experiment by means of an external water bath. The concentration of calcein in the receiving liquid was then monitored by withdrawing aliquots from arm 50 and analysing them. This experiment was conducted under three sets of conditions:

1) in the absence of microneedles (“without enhancers” in FIG. 9)
2) with microneedles but with no ultrasound (“microneedles” in FIG. 9)
3) with microneedles and ultrasound (20 kHz, 0.5 Wcm⁻²) (“microneedles+US” in FIG. 9).

From the results in FIG. 9 it can be seen that microneedles alone provide an improvement in transport of calcein across the skin sample, however this is enhanced by application of ultrasound.

1. An injection device for injecting a substance into a subject, said injection device comprising:
(i) an injector comprising a microneedle support and at least one microneedle extending from a first surface of said support, wherein the or each microneedle has a fluid channel extending through the microneedle and through the support, the fluid channel of the or each microneedle having an inlet aperture in a second surface of the support and an outlet aperture in the microneedle; and
(ii) an enhancer for enhancing the rate of penetration of the substance into the subject.

2. The injection device according to claim 1 wherein the enhancer comprises an ultrasound generator.

3. The injection device of claim 2 wherein the ultrasound generator comprises a piezoelectric crystal.

4. The injection device according to claim 3 wherein the piezoelectric crystal comprises a PZT membrane.

5. The injection device of claim 3 or claim 4 wherein the piezoelectric crystal is coupled to a source of alternating current whereby the piezoelectric crystal is capable of generating ultrasound in response to an alternating current from said source.

6. The injection device of any one of claims 1 to 5 additionally comprising a reservoir for containing a fluid comprising the substance, said reservoir being disposed so as to allow the fluid to pass from the reservoir into the or each fluid channel through the inlet aperture thereof.

7. The injection device of claim 6 wherein the enhancer is coupled to the microneedle support so as to define the reservoir between the second surface of the support and the enhancer.

8. The injection device of any one of claims 1 to 7 wherein the injector is made of silicon.

9. The injection device of any one of claims 1 to 8 wherein the or each microneedle has a length such that, in use, it penetrates through the stratum corneum of the subject and does not penetrate to the dermis of the subject, so as to deliver the substance into the epidermis of the subject.

10. The injection device of any one of claims 1 to 9 wherein the microneedle, or each microneedle independently, is between about 50 and about 150 microns long.

11. The injection device of any one of claims 1 to 10 wherein the at least one microneedle is an array of microneedles, each of said microneedles being capable of penetrating to the epidermis of the subject so as to inject the substance into the epidermis.

12. The injection device of claim 11 wherein the array comprises between about 500 and about 2000 microneedles.

13. A method for delivering a substance to a subject, said method comprising:
providing an injection device for injecting the substance into the subject, said injection device comprising: (i) an injector comprising a microneedle support and at least one microneedle extending from a first surface of said support, wherein the or each microneedle has a fluid channel extending through the microneedle and through the support, the fluid channel of the or each microneedle having an inlet aperture in a second surface of the support and an outlet aperture in the microneedle; and (ii) an enhancer for enhancing the rate of penetration of the substance into the subject;
applying the injector to the skin of the subject so that the at least one microneedle penetrates the skin of the subject;
supplying a fluid comprising the substance to the or each inlet aperture so as to allow said fluid to enter the fluid channel or channels; and
activating the enhancer so as to enhance the rate of penetration of the substance into the subject.

14. The method of claim 13 wherein the enhancer comprises an ultrasound generator and the step of activating the enhancer comprises causing the ultrasound generator to supply ultrasound to the skin of the subject adjacent to the or each microneedle.

15. The method of claim 13 or claim 14 wherein the injection device comprises a reservoir for containing the fluid comprising the substance, said reservoir being disposed so as to allow the fluid to pass from the reservoir into the or each fluid channel through the inlet aperture thereof, and the step of supplying the fluid comprises supplying the fluid to the reservoir.

16. The method of any one of claims 13 to 15 wherein the substance is a therapeutic substance indicated for treating a condition of the subject, whereby the method is a method for treating the condition and the step of supplying the fluid comprises supplying a fluid containing a therapeutically effective dose of the substance to the aperture or apertures.

17. A process for making an injection device for injecting a substance into a subject, said process comprising coupling an injector to an enhancer for enhancing the rate of penetration of a substance into a subject, said injector comprising a microneedle support and at least one microneedle extending from a first surface of said support, wherein the or each microneedle has a fluid channel extending through the microneedle and through the support, the fluid channel of the or each microneedle having an inlet aperture in a second surface of the support and an outlet aperture in the microneedle.

18. The process of claim 17 wherein the enhancer is an ultrasound generator, and the process additionally comprises the step of coupling a source of alternating current to said ultrasound generator.

19. The process of claim 17 or 18 wherein the step of coupling the injector to the enhancer comprises coupling the microneedle support to the enhancer so as to form a reservoir between the second surface of the support and the enhancer.

20. The process of claim 19 wherein the step of coupling the microneedle support to the enhancer comprises coupling a spacer to the second surface of the microneedle support and coupling the enhancer to the spacer.

21. The process of any one of claims 17 to 20 comprising the step of fabricating the one or more microneedles from a silicon wafer using microfabrication techniques.

22. An injection device made by the process of any one of claims 17 to 21.

23. An injection device according to any one of claims 1 to 12 or 22 when used for injecting a substance into a subject.

24. Use of an injection device according to any one of claims 1 to 12 or 22 for injecting a substance into a subject.
